(19) United States
(12) Patent Application Publication
(10) Pub. No.: US 2025/0120707 A1
(43) Pub. Date: Apr. 17, 2025

(54) SURGICAL INSTRUMENTS WITH LATERALLY ENGAGEABLE LOCKING ARRANGEMENTS FOR LOCKING A FIRING ACTUATOR

(71) Applicant: Cilag GmbH International, Zug (CH)

(72) Inventors: Frederick E. Shelton, IV, Hillsboro, OH (US); Jason L. Harris, Lebanon, OH (US)

(21) Appl. No.: 18/379,815

(22) Filed: Oct. 13, 2023

(51) Int. Cl. A61B 17/072; A61B 17/29

(52) U.S. Cl. CPC A61B 17/072; A61B 2017/07257; A61B 2017/07271; A61B 2017/07285; A61B 2017/2933; A61B 2017/2946

(57) ABSTRACT

A surgical stapler is disclosed comprising a firing beam, a firing bar movable to transition the firing beam from a proximal position toward a distal position, and a firing lockout comprising a first lockout arm extending on a first lateral side of the firing bar. The firing bar comprises a plurality of laminated strips. The plurality of laminated strips comprises a central laminated strip and a first subset of laminated strips. A first notch is defined in the first subset of laminated strips. The first lockout arm comprises a first projection. The firing lockout is configurable between a locked state in which the first projection is received in the first notch to prevent the firing beam from moving toward the distal position and an unlocked state in which the first projection is displaced from the first notch to permit the firing beam to move toward the distal position.

### BACKGROUND

The present invention relates to surgical instruments and, in various arrangements, to surgical stapling and cutting instruments and staple cartridges for use therewith that are designed to staple and cut tissue.

### SUMMARY

In accordance with the present disclosure, lockouts are utilized with surgical stapling and cutting instruments to ensure that certain conditions are met prior to allowing a clinician to cut and staple tissue with the surgical stapling and cutting instrument. These lockouts ensure that patient tissue is not inadvertently cut and/or stapled prior to these conditions being met.

In accordance with the present disclosure, a surgical stapler is disclosed comprising an end effector comprising an elongate channel, a firing beam, a firing bar movable to transition the firing beam from a proximal position toward a distal position during a firing stroke, and a firing lockout comprising a first lockout arm extending on a first lateral side of the firing bar. The firing bar comprises a plurality of laminated strips. The plurality of laminated strips comprises a central laminated strip and a first subset of laminated strips extending on a first lateral side of the central laminated strip. A first notch is defined in the first subset of laminated strips. The first lockout arm comprises a first projection. The firing lockout is configurable between a locked state in which the first projection is received in the first notch to prevent the firing beam from moving toward the distal position and an unlocked state in which the first projection is displaced from the first notch to permit the firing beam to move toward the distal position.

In accordance with the present disclosure, a surgical stapler is disclosed comprising an end effector comprising a staple cartridge comprising a plurality of staples removably stored therein, a firing beam, a firing bar movable to transition the firing beam from a proximal position toward a distal position during a firing stroke, and a firing lockout. The firing bar comprises a plurality of laminated strips. The plurality of laminated strips comprises a central laminated strip, a first subset of laminated strips extending on a first lateral side of the central laminated strip, and a second subset of laminated strips extending on a second lateral side of the central laminated strip. A first notch is defined in the first subset of laminated strips. A second notch is defined in the second subset of laminated strips. The firing lockout comprises a body, a first lockout arm extending from the body on a first lateral side of the firing bar, and a second lockout arm extending from the body on a second lateral side of the firing bar. The first lockout arm comprises a first projection. The second lockout arm comprises a second projection. The firing lockout is configurable between a locked state in which the first projection is received in the first notch and the second projection is received in the second notch to prevent the firing beam from moving toward the distal position and an unlocked state in which the first projection is displaced from the first notch and the second projection is displaced from the second notch to permit the firing beam to move toward the distal position. The staple cartridge further comprises a first key configured to engage the first lockout arm to displace the first projection from the first notch and a second key configured to engage the second lockout arm to displace the second projection from the second notch, wherein the first key and the second key transition the firing lockout to the unlocked state.

In accordance with the present disclosure, a surgical stapler is disclosed comprising an end effector, a firing beam, a firing bar movable to transition the firing beam from a proximal position toward a distal position during a firing stroke, and a firing lockout. The end effector comprises a first jaw and a second jaw movable relative to the first jaw between an open position and a closed position. The firing bar comprises a plurality of laminated strips. An aperture is defined through the plurality of laminated strips. The firing lockout comprises a first lockout arm extending on a first lateral side of the firing bar and a second lockout arm extending on a second lateral side of the firing bar. The first lockout arm comprises a first projection. The second lockout arm comprises a second projection. The firing lockout is configurable between a locked state in which the first projection and the second projection extend within the aperture to prevent the firing beam from moving toward the distal position and an unlocked state in which the first projection and are second projection are displaced from the aperture to prevent the firing beam from moving toward the distal position.

### LISTING OF THE FIGURES

Various features of the embodiments described herein, together with advantages thereof, may be understood in accordance with the following description taken in conjunction with the accompanying drawings as follows:

FIG. 1 is a partial perspective view of a surgical stapler including an end effector;

FIG. 2 is an exploded perspective view of the end effector and staple cartridge of FIG. 1;

FIG. 3 is another perspective view of the end effector of FIG. 1 with the staple cartridge removed;

FIG. 4 is an elevational view of the end effector of FIG. 1 in a closed position;

FIG. 5 is an elevational view of the end effector of FIG. 1 in an open position;

FIG. 6 is a partial perspective view of the staple cartridge, a firing assembly, and a lockout of the surgical stapler of FIG. 1;

FIG. 7 is a perspective view of the lockout of FIG. 6; and

FIG. 8 is a top cross-sectional view of the staple cartridge, the firing assembly, and the lockout of FIG. 6.

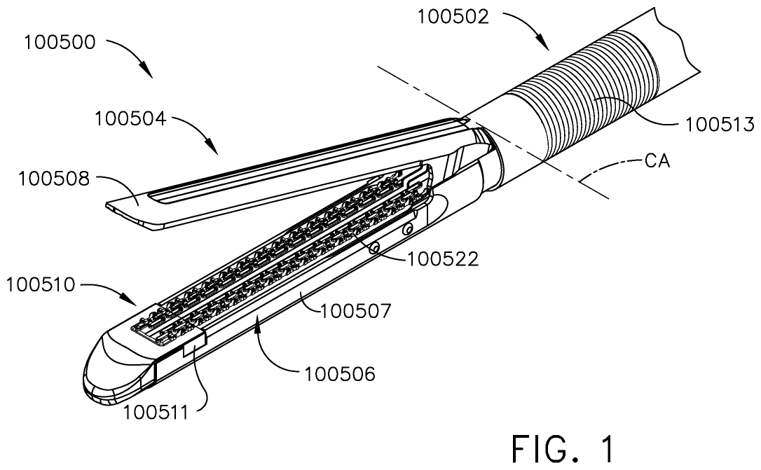

Corresponding reference characters indicate corresponding parts throughout the several views.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working frame through which the end effector and elongate shaft of a surgical instrument can be advanced.

Various mechanical lockouts are utilized with surgical staplers to prevent erroneous firings that include, but not limited to, firing a spent staple cartridge, firing an unauthorized staple cartridge, firing a non-compatible staple cartridge, and/or firing without a staple cartridge. Some mechanical lockouts are configured to interfere with the firing stroke, for example by preventing advancement of a firing assembly of a surgical stapler, in the event of an erroneous firing. To prevent advancement of the firing assembly, the lockouts engage components of the firing assembly, e.g. firing bar or firing beam, at positions that are offset from the firing access. Consequently, attempting to advance the firing assembly against the resistance of the lockout can yield a blowout, due to the torque applied to the lockout. As discussed below in greater detail, the present disclosure can mitigate firing assembly blowouts by utilizing novel and unique lockouts that are selectively configured to reduce, or eliminate, the amount of leverage that the firing assembly can apply to the lockouts.

Referring now to the Figures, a surgical stapler 100500 is shown that includes a shaft 100502 and an end effector 100504 extending from the shaft 100502. The end effector 100504 includes a first jaw 100506 and a second jaw 100508. The first jaw 100506 includes an elongate channel 100507 and a staple cartridge 100510. The staple cartridge 100510 is insertable into and removable from the elongate channel 100507. In particular, the staple cartridge 100510 includes tabs 100511 on each lateral side thereof that snap into corresponding recesses 100509 defined in the elongate channel 100507. Alternatively, the staple cartridge 100510 may be integral with, or not removable from, the first jaw 100506.

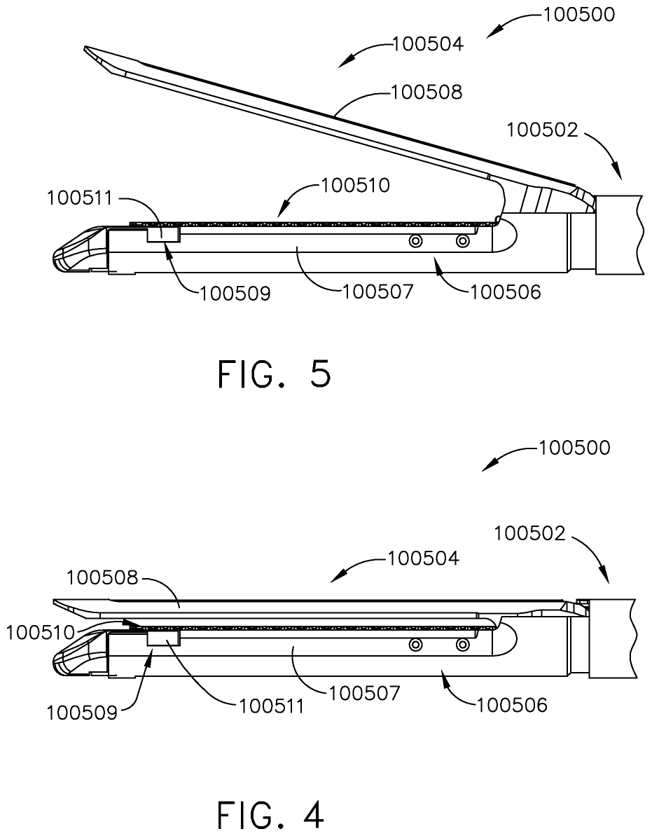

The second jaw 100508 includes an anvil configured to deform staples ejected from the staple cartridge 100510. The second jaw 100508 is pivotable relative to the first jaw 10056 about a closure axis CA between an open position (FIG. 5) and a closed position (FIG. 4); however, the first jaw 100506 may also be pivotable relative to the second jaw 100508. The surgical stapler 100500 further includes an articulation joint 100513 configured to permit the end effector 100504 to be rotated, or articulated, relative to the shaft 100502. Alternatively, the surgical stapler may not include an articulation joint.

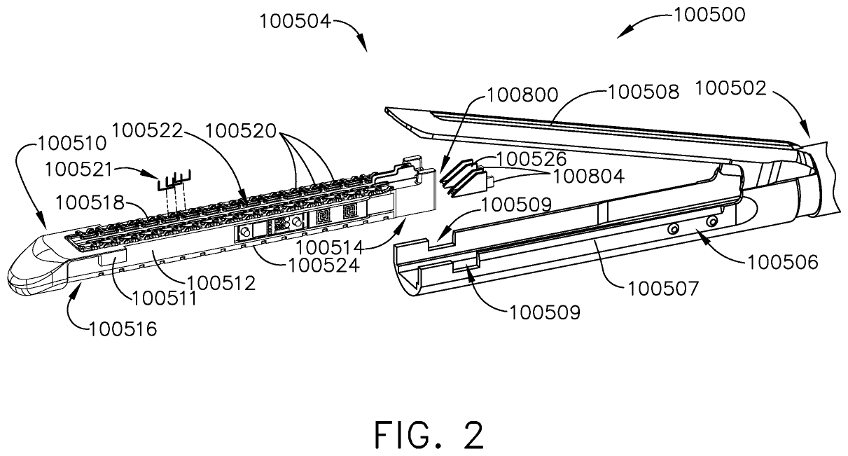

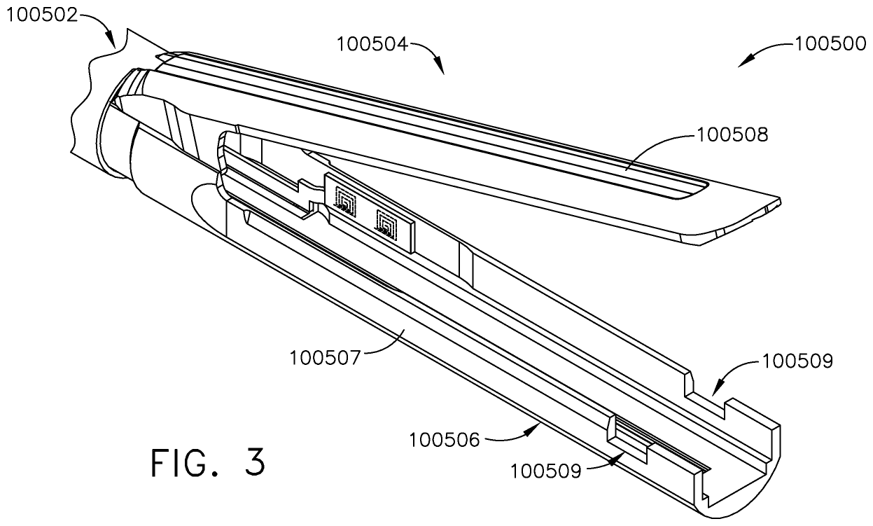

As shown in FIG. 2, the staple cartridge 100510 includes a cartridge body 100512. The cartridge body 100512 includes a proximal end 100514, a distal end 100516, and a deck 100518 extending between the proximal end 100514 and the distal end 100516. In use, the staple cartridge 100510 is positioned on a first side of the tissue to be stapled and the anvil 100508 is positioned on a second side of the tissue. The anvil 100508 is moved toward the staple cartridge 100510 (see FIGS. 4 and 5) to compress and clamp the tissue against the deck 100518. Thereafter, staples 100521 removably stored in the cartridge body 100512 can be deployed into the tissue. The cartridge body 100512 includes staple cavities 100520 defined therein wherein the staples 100521 are removably stored in the staple cavities 100520. The staple cavities 100520 are arranged in six longitudinal rows. Three rows of staple cavities are positioned on a first side of a longitudinal slot 100522 and three rows of staple cavities are positioned on a second side of the longitudinal slot 100522. Other arrangements of staple cavities and staples may be possible.

The staples are supported by staple drivers in the cartridge body 100512. Staples supported on staple drivers can be seen in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, filed Sep. 14, 2020, which is hereby incorporated by reference in its entirety herein. The drivers are movable between a first, or unfired position, and a second, or fired, position to eject the staples from the staple cavities 100520. The drivers are retained in the cartridge body 100512 by a retainer 100524 which extends around the bottom of the cartridge body 100512 and includes resilient members configured to grip the cartridge body 100512 and hold the retainer 100524 to the cartridge body 100512. The drivers are movable between their unfired positions and their fired positions by a sled 100526. The sled 100526 is movable between a proximal position adjacent the proximal end 100514 and a distal position adjacent the distal end 100516. The sled 100526 includes a plurality of ramped surfaces configured to slide under the drivers and lift the drivers, and the staples 100521 supported thereon, toward the anvil 100508. The staples 100521 may not be supported by staple drivers, but rather, the staples include integral drive surfaces that are directly engaged by the sled 100526 to lift the staples, examples of which are described in U.S. Patent Application Publication No. 2015/0173756, entitled SURGICAL CUTTING AND STAPLING METHODS, filed Dec. 23, 2013, which is hereby incorporated by reference in its entirety herein.

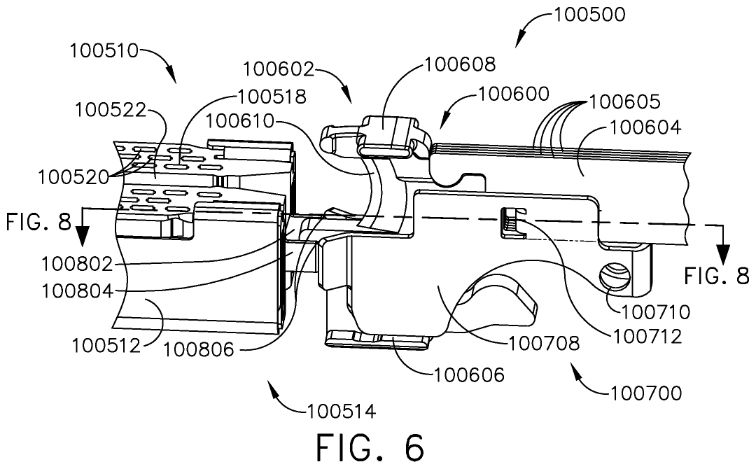

Further to the above, the sled 100526 is moved distally by a firing assembly 100600 (see FIG. 6) that includes a firing member or firing beam 100602 and a firing bar 100604 extending proximally from the firing beam 100602. The firing beam 100602 is configured to contact the sled 100526 and push the sled 100526 toward the distal end 100516 during a firing stroke. The firing bar 100604 includes a plurality of laminate strips 100605. The longitudinal slot 100522 defined in the cartridge body 100512 is configured to accommodate the firing beam 100602 and the firing bar 100604 during the firing stroke of the firing assembly 100600. The anvil 100508 also includes a slot defined therein that receives the firing beam 100602. The firing beam 100602 further includes a first cam 100606 which engages the first jaw 100506 and a second cam 100608 which engages the second jaw 100508 in the slot of the anvil. As the firing beam 100602 is advanced distally through the firing stroke, the first cam 100606 and the second cam 100608 control the distance, or tissue gap, between the deck 100518 of the staple cartridge 100510 and the anvil 100508. The firing beam 100602 also includes a knife 100610 configured to incise the tissue captured intermediate the staple cartridge 100510 and the anvil 100508. It is desirable for the knife 100610 to be positioned at least partially proximal to the ramped surfaces such that the staples are ejected ahead of the knife 100610.

Closure of the end effector 100504 can be performed separately and distinctly from the firing of the staples 100521. As such, the anvil 100508 includes a ramp on a proximal end thereof, the ramp engageable by a closure tube that is movable distally to cam the anvil 100508 to the closed position. To transition the anvil 100508 to the open position, the closure tube is retracted proximally, and springs positioned within the end effector 100504 bias the anvil 100508 to the open position. The anvil 100508 may include a tab and the closure tube defines an aperture which engages the tab as the closure tube moves proximally, thereby positively transitioning the anvil 100508 to the open position. Exemplary closure mechanisms are described in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, filed Sep. 14, 2020, which is hereby incorporated by reference in its entirety herein.

The anvil 100508 can be moved from the open position to the closed position using the firing beam 100602. As such, the anvil 100508 may include a ramp that extends proximally from the slot defined therein that is engaged by the second cam 100608 of the firing beam 100602 during a first, closure stroke portion of the firing beam 100602 to move the anvil 100508 to the closed position. The first closure stroke portion of the firing beam can be about 0.105″-0.250″ of the firing beam 100602 travel. At the end of the first closure stroke portion of the firing beam 100602, the firing beam 100602 can continue advancing distally through a second firing stroke portion to deploy staples from the staple cartridge 100510 and incise tissue captured by the end effector with the knife 100610, as described above. Exemplary firing beams that close the anvil and fire staples are described in U.S. Pat. No. 11,160,551, entitled ARTICULATABLE SURGICAL STAPLING INSTRUMENTS, filed Dec. 21, 2016, which is hereby incorporated by reference in its entirety herein. When a firing beam is utilized to move the anvil 100508 toward the closed position and deploy staples from the staple cartridge 100510, the firing beam can be considered both a closure system and/or a firing assembly.

After a staple cartridge 100510 has been fired, or at least partially fired, it is removed from the elongate channel 100507 and then replaced with another replaceable staple cartridge, if desired. At such point, the surgical stapler 100500 can be re-used to continue stapling and incising the patient tissue. In some instances, however, a previously-fired staple cartridge can be accidentally loaded into the elongate channel 100507. If the firing beam 100602 were to be advanced distally within such a previously-fired staple cartridge, the surgical stapler 100500 would cut the patient tissue without stapling it. The surgical stapler 100500 would similarly cut the patient tissue without stapling it if the firing beam 100602 were advanced distally through a staple firing stroke without a staple cartridge positioned in the elongate channel 100507 at all. In addition, various surgical staple cartridges may have different arrays of and/or orientations of staples/fasteners therein. The sizes of the staples or fasteners, as well as the number of fasteners may vary from cartridge type to cartridge type depending upon a particular surgical procedure or application. To ensure that the staples are properly crimped or formed, the surgical staple cartridges must be used in connection with corresponding, compatible anvils that have the proper array of staple-forming pockets therein as well as the proper cutting and firing components. Should a “non-compatible” cartridge be loaded into a surgical stapling device that has an anvil that is mismatched to the staple cartridge, the staples may not be properly formed during the firing process.

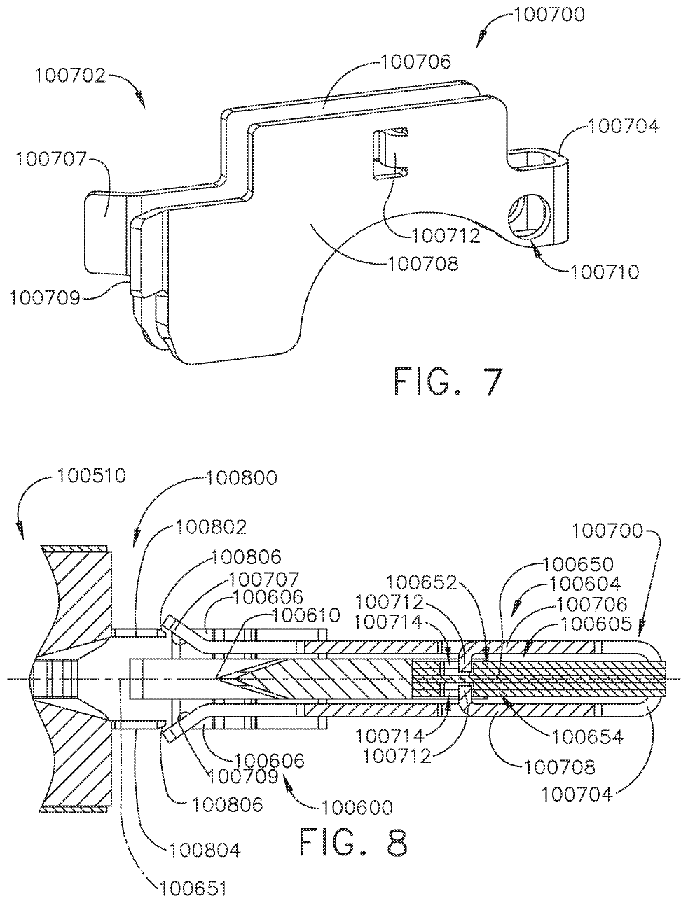

To this end, the surgical stapler 100500 further includes a lockout 100700 that is configured to prevent the firing assembly 100600 from moving distally through the firing stroke unless an authorized or compatible staple cartridge is operably seated in the elongate channel 100507. Referring to FIGS. 6 and 7, the lockout 100700 includes a single, bi-lateral lockout spring 100702. The spring 100702 can be supported, or at least partially supported, in the elongate channel 100507. The spring 100702 can be supported, or at least partially supported, in the shaft 100502. The spring 100702 can be attached to a shaft mount flange, similar to the shaft mount flange discussed in U.S. Pat. No. 11,298,129, entitled METHOD FOR PROVIDING AN AUTHENTICATION LOCKOUT IN A SURGICAL STAPLER WITH A REPLACEABLE CARTRIDGE, filed Jun. 26, 2019, which is hereby incorporated by reference in its entirety herein. The spring 100702 can be directly attached to the elongate channel 100507.

The lockout spring 100702 includes a central body portion 100704, a first lockout arm 100706 extending from a first side of the central body portion 100704, and a second lockout arm 100708 extending from a second side of the central body portion 100704. The spring 100702 is supported in the elongate channel 100507 and affixed to the shaft mount flange or elongate channel 100507 by a pin that extends through holes in the shaft mount flange or elongate channel 100507 and through holes 100710 in the first lockout arm 100706 and the second lockout arm 100708. As seen in FIG. 6, the firing bar 100604 extends above the central body portion 100704.

The first lockout arm 100706 and the second lockout arm 100708 include projections 100712 that extend transversely therefrom. The projections 100712 are receivable in corresponding notches 100714 defined in the plurality of laminate strips 100605. The plurality of laminated strips 100605 may include a central laminated strip 100650 that defines a firing bar axis 100651, a first subset of laminated strips 100652 that extend along a first lateral side of the central laminated strip 100650, and a second subset of laminated strips 100654 that extend along a second lateral side of the central laminated strip 100650. The notch 100714 that receives the projection 100712 from the first lockout arm 100706 can extend through some, or all, of the first subset of laminated strips 100652. Similarly, the notch 100714 that receives the projection 100712 from the second lockout arm 100708 extends through some, or all, of the second subset of laminated strips 100654. As such, a notch may be absent from the central laminated strip 100650, thus preventing the tips of the projections 100712 from engaging one another. A notch can also be defined in the central laminated strip 100650 such that an aperture is defined through all of the plurality of laminated strips 100605. As such, the projection 100712 from the first lockout arm 100706 and the projection 100712 from the second lockout arm 100708 may extend within the aperture and engage one another at, or around, the firing bar axis 100651.

As referenced above, the firing lockout 100700 is configured to prevent the firing assembly 100600 from moving distally through the firing stroke unless an authorized or compatible staple cartridge is operably seated in the elongate channel 100507. The firing lockout 100700 is configurable between a locked state and an unlocked state. In the locked state, the projection 100712 from the first lockout arm 100706 is received in the notch 100714 in the first subset of laminated strips 100652 and the projection 100712 from the second lockout arm 100708 is received in the notch 100714 in the second subset of laminated strips 100654, as shown in FIGS. 6 and 8. In the locked state, the projections 100712 engage the firing bar 100604 to prevent distal movement of the firing assembly 100600. In the unlocked state, the projection 100712 from the first lockout arm 100706 is pivoted laterally away, or displaced, from the notch 100714 in the first subset of laminated strips 100652 and the projection 100712 from the second lockout arm 100708 is pivoted laterally away, or displaced, from, the notch 100714 in the second subset of laminated strips 100654. In the unlocked state, the projections 100712 permit distal movement distal movement of the firing assembly 100600.

As referenced above, the firing lockout 100700 includes a lockout spring 100702 that includes a central body portion 100704, a first lockout arm 100706 extending from a first side of the central body portion 100704, and a second lockout arm 100708 extending from a second side of the central body portion 100704. In a rested state of the lockout spring 100702, the projections 100712 of the first lockout arm 100706 and the second lockout arm 100708 are received in their corresponding notches 100714. Stated another way, the lockout spring 100702 naturally rests in the locked state. As the lockout 100700 is transitioned to the unlocked state, the projections 100712 are pivoted or otherwise moved away from their corresponding notches 100714, and therefore, the first lockout arm 100706 and second lockout arm 100708 are pivoted relative to the central body portion 100704. Given the inherent nature of springs, the first lockout arm 100706 and second lockout arm 100708 are naturally biased toward the resting state of the firing lockout 100700, and therefore, the firing lockout 100700 is naturally biased toward the locked state.

Lockouts that engage the firing beam at locations that are laterally outward from the firing bar axis 100651 may be subject to “blowout”, due to an amount of torque that is applied by the firing assembly to the lockout. Shifting the engagement location between the lockout 100700 and the firing assembly 100600 to, or close to, the firing bar axis 100651 reduces, or eliminates, the amount of leverage that the firing assembly 100600 can apply to the lockout 100700, thus decreasing the chance of blowout of the lockout 100700 from occurring.

As discussed above, the lockout 100700 prevents the firing beam from moving distally from its proximal-most, starting position unless an authorized or compatible staple cartridge is operably seated in the elongate channel 100507. An authorized or compatible staple cartridge can include a release key 100800 that is configured to transition the lockout 100700 from the locked state to the unlocked state.

As shown FIG. 6, the authorized or compatible staple cartridge 100510 can include a release key 100800 that includes a first key 100802 and a second key 100804. As shown more clearly in FIG. 2, the sled 100526 can include the first key 100802 and the second key 100804. As such, the first key 100802 and the second key 100804 can be sized to engage the lockout 100700 when the sled 100526 is in its proximal, unfired position, which is indicative of an unfired, or unspent, state cartridge (i.e., no staples have been deployed from the staple cartridge). Accordingly, positioning the release key 100800 on the sled 100526 both informs a clinician that, not only is the staple cartridge 100510 an authorized or compatible staple cartridge, but also that the staple cartridge 100510 is unspent and ready for firing. The release key 100800 can be integrally molded with the cartridge body 100512. Alternatively, the retainer 100524 can include the release key 100800. If the cartridge body 100512 or the retainer 100524 include the release key 100800, the release key 100800 can be breakable from the staple cartridge by the clinician once the staple cartridge has been fired to ensure that lockout 100700 prevents a subsequent actuation of the firing assembly through the spent stable cartridge. The keys 100802, 100804 include tips 100806.

Referring again to FIG. 7, the first lockout arm 100706 includes a first cam surface 100707 on a distal end thereof and the second lockout arm 100708 includes a second cam surface 100709 on a distal end thereof. In operation, the authorized or compatible staple cartridge 100510 may be inserted, e.g. longitudinally, into the elongate channel 100507 so as to bring the tip 100806 of the first key 100802 into contact with the first cam surface 100707 on the first lockout arm 100706 and the tip 100806 of the second key 100804 into contact with the second cam surface 100709 on the second lockout arm 100708.

During the insertion of the authorized or compatible staple cartridge 100510 in a proximal direction into the elongate channel 100507, the tips 100806 on the first key 100802 and the second key 100804 pivot the first lockout arm 100706 and the second lockout arm 100708 of the lockout spring 100702 laterally outward, in opposite directions, causing the projections 100712 to pivot out of the corresponding notches 100714, transitioning the firing lockout 100700 to an unlocked state. After the firing lockout 100700 has been transitioned to the unlocked state, a user is free to actuate the firing assembly 100600 and drive the firing assembly through its firing stroke.

Accordingly, the firing lockout 100700 prevents the firing assembly 100600 from advancing through its firing stroke unless a compatible staple cartridge is positioned in the elongate channel 100507. As referenced above, the firing lockout 100700 provides the benefit of shifting the point of engagement between the firing lockout 100700 and the firing assembly 100600 at, or close to, the firing bar axis 100651 of the firing bar 100604, which reduces, or eliminates, the chance of the firing lockout 100700 blowing out when an incompatible staple cartridge is positioned in the elongate channel 100507.

While the firing lockout 100700 shown and described above includes two lockout arms 100706, 100708 for preventing movement of the firing assembly 100600 unless a compatible staple cartridge is positioned in the elongate channel 100507, the firing lockout 100700 may only includes one lockout arm for preventing movement of the firing assembly 100600. As such, the firing lockout 100700 may require only one key on the staple cartridge to transition the lockout arm from the unlocked position to the locked position.

Many of the surgical instrument systems described herein are motivated by an electric motor; however, the surgical instrument systems described herein can be motivated in any suitable manner. The surgical instrument systems described herein can be motivated by a manually-operated trigger, for example. The motors disclosed herein may include a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

Various aspects of the subject matter described herein are set out in the following numbered examples:

Example 1—A surgical stapler (100500) comprising an end effector (100504) comprising an elongate channel (100507), a firing beam (100602), a firing bar (100604) movable to transition the firing beam from a proximal position toward a distal position during a firing stroke, and a firing lockout (100700) comprising a first lockout arm (100706) extending on a first lateral side of the firing bar. The firing bar comprises a plurality of laminated strips (100605). The plurality of laminated strips comprises a central laminated strip (100650) and a first subset of laminated strips (100652) extending on a first lateral side of the central laminated strip. A first notch (100714) is defined in the first subset of laminated strips. The first lockout arm comprises a first projection (100712). The firing lockout is configurable between a locked state in which the first projection is received in the first notch to prevent the firing beam from moving toward the distal position and an unlocked state in which the first projection is displaced from the first notch to permit the firing beam to move toward the distal position.

Example 2—The surgical stapler of Example 1, wherein the plurality of laminated strips further comprises a second subset of laminated strips (100654) extending on a second lateral side of the central laminated strip, and wherein a second notch (100714) is defined in the second subset of laminated strips.

Example 3—The surgical stapler of Example 2, wherein the firing lockout further comprises a second lockout arm (100708) extending on a second lateral side of the firing bar, and wherein the second lockout arm comprises a second projection (100712).

Example 4—The surgical stapler of Example 3, wherein in the locked state of the firing lockout, the second projection is received in the second notch to prevent the firing beam from moving toward the distal position and in the unlocked state of the firing lockout, the second projection is displaced from the second notch to permit the firing beam to move toward the distal position.

Example 5—The surgical stapler of Example 3, wherein the firing lockout further comprises a central body portion (100704), and wherein the first lockout arm and the second lockout arm extend from the central body portion.

Example 6—The surgical stapler of Example 5, wherein the firing bar extends above the central body portion.

Example 7—The surgical stapler of any one of Example 1-6, further comprising a staple cartridge (100510) comprising a key (100802), wherein the staple cartridge is removably positionable in the elongate channel, and wherein the key is configured to engage the first lockout arm to transition the firing lockout to the unlocked state.

Example 8—The surgical stapler of Example 7, wherein the first lockout arm comprises a cam surface (100707), and wherein the key is configured to engage the cam surface to transition the firing lockout to the unlocked state.

Example 9—The surgical stapler of Example 7, wherein the staple cartridge comprises a cartridge body (100512), and wherein the cartridge body comprises the key.

Example 10—The surgical stapler of Example 7, wherein the staple cartridge comprises a sled (100526), and wherein the sled comprises the key.

Example 11—The surgical stapler of Example 7, wherein the staple cartridge comprises a retainer (100524), and wherein the retainer comprises the key.

Example 12—The surgical stapler of Example 7, wherein the end effector further comprises an anvil (100508) movable relative to the elongate channel between an open position and a closed position, and wherein the firing stroke comprises a first stroke portion in which the firing beam moves the anvil toward the closed position and a second stroke portion in which the firing beam deploys staples from the staple cartridge.

Example 13—The surgical stapler of any one of Example 1-6, wherein the firing lockout comprises a lockout spring (100702), and wherein the lockout spring is naturally biased toward the locked state.

Example 14—The surgical stapler of any one of Example 1-6, wherein the end effector further comprises an anvil (100508), and wherein the firing beam comprises a first cam (100606) configured to engage the elongate channel during the firing stroke and a second cam (100608) configured to engage the anvil during the firing stroke.

Example 15—The surgical stapler of any one of Example 1-6, wherein the firing beam comprises a knife (100610).

Example 16—A surgical stapler comprising an end effector comprising an elongate channel, a firing beam, a firing bar movable to transition the firing beam from a proximal position toward a distal position during a firing stroke, and a firing lockout comprising a first lockout arm extending on a first lateral side of the firing bar. The firing bar comprises a plurality of laminated strips. The plurality of laminated strips comprises a central laminated strip and a first subset of laminated strips extending on a first lateral side of the central laminated strip. A first notch is defined in the first subset of laminated strips. The first lockout arm comprises a first projection. The firing lockout is configurable between a locked state in which the first projection is received in the first notch to prevent the firing beam from moving toward the distal position and an unlocked state in which the first projection is displaced from the first notch to permit the firing beam to move toward the distal position.

Example 17—The surgical stapler of Example 16, wherein the plurality of laminated strips further comprises a second subset of laminated strips extending on a second lateral side of the central laminated strip, and wherein a second notch is defined in the second subset of laminated strips.

Example 18—The surgical stapler of Example 17, wherein the firing lockout further comprises a second lockout arm extending on a second lateral side of the firing bar, and wherein the second lockout arm comprises a second projection.

Example 19—The surgical stapler of Example 18, wherein in the locked state of the firing lockout, the second projection is received in the second notch to prevent the firing beam from moving toward the distal position and in the unlocked state of the firing lockout, the second projection is displaced from the second notch to permit the firing beam to move toward the distal position.

Example 20—The surgical stapler of Example 18, wherein the firing lockout further comprises a central body portion, and wherein the first lockout arm and the second lockout arm extend from the central body portion.

Example 21—The surgical stapler of Example 20, wherein the firing bar extends above the central body portion.

Example 22—The surgical stapler of Example 16, further comprising a staple cartridge comprising a key, wherein the staple cartridge is removably positionable in the elongate channel, and wherein the key is configured to engage the first lockout arm to transition the firing lockout to the unlocked state.

Example 23—The surgical stapler of Example 22, wherein the first lockout arm comprises a cam surface, and wherein the key is configured to engage the cam surface to transition the firing lockout to the unlocked state.

Example 24—The surgical stapler of Example 22, wherein the staple cartridge comprises a cartridge body, and wherein the cartridge body comprises the key.

Example 25—The surgical stapler of Example 22, wherein the staple cartridge comprises a sled, and wherein the sled comprises the key.

Example 26—The surgical stapler of Example 22, wherein the staple cartridge comprises a retainer, and wherein the retainer comprises the key.

Example 27—The surgical stapler of Example 22, wherein the end effector further comprises an anvil movable relative to the elongate channel between an open position and a closed position, and wherein the firing stroke comprises a first stroke portion in which the firing beam moves the anvil toward the closed position and a second stroke portion in which the firing beam deploys staples from the staple cartridge.

Example 28—The surgical stapler of Example 16, wherein the firing lockout comprises a lockout spring, and wherein the lockout spring is naturally biased toward the locked state.

Example 29—The surgical stapler of Example 16, wherein the end effector further comprises an anvil, and wherein the firing beam comprises a first cam configured to engage the elongate channel during the firing stroke and a second cam configured to engage the anvil during the firing stroke.

Example 30—The surgical stapler of Example 16, wherein the firing beam comprises a knife.

Example 31—A surgical stapler comprising an end effector comprising a staple cartridge comprising a plurality of staples removably stored therein, a firing beam, a firing bar movable to transition the firing beam from a proximal position toward a distal position during a firing stroke, and a firing lockout. The firing bar comprises a plurality of laminated strips. The plurality of laminated strips comprises a central laminated strip, a first subset of laminated strips extending on a first lateral side of the central laminated strip, and a second subset of laminated strips extending on a second lateral side of the central laminated strip. A first notch is defined in the first subset of laminated strips. A second notch is defined in the second subset of laminated strips. The firing lockout comprises a body, a first lockout arm extending from the body on a first lateral side of the firing bar, and a second lockout arm extending from the body on a second lateral side of the firing bar. The first lockout arm comprises a first projection. The second lockout arm comprises a second projection. The firing lockout is configurable between a locked state in which the first projection is received in the first notch and the second projection is received in the second notch to prevent the firing beam from moving toward the distal position and an unlocked state in which the first projection is displaced from the first notch and the second projection is displaced from the second notch to permit the firing beam to move toward the distal position. The staple cartridge further comprises a first key configured to engage the first lockout arm to displace the first projection from the first notch and a second key configured to engage the second lockout arm to displace the second projection from the second notch, wherein the first key and the second key transition the firing lockout to the unlocked state.

Example 32—The surgical stapler of Example 31, wherein the staple cartridge further comprises a sled, and wherein the sled comprises the first key and the second key.

Example 33—A surgical stapler comprising an end effector, a firing beam, a firing bar movable to transition the firing beam from a proximal position toward a distal position during a firing stroke, and a firing lockout. The end effector comprises a first jaw and a second jaw movable relative to the first jaw between an open position and a closed position. The firing bar comprises a plurality of laminated strips. An aperture is defined through the plurality of laminated strips. The firing lockout comprises a first lockout arm extending on a first lateral side of the firing bar and a second lockout arm extending on a second lateral side of the firing bar. The first lockout arm comprises a first projection. The second lockout arm comprises a second projection. The firing lockout is configurable between a locked state in which the first projection and the second projection extend within the aperture to prevent the firing beam from moving toward the distal position and an unlocked state in which the first projection and are second projection are displaced from the aperture to prevent the firing beam from moving toward the distal position.

Example 34—The surgical stapler of Example 33, further comprising a staple cartridge comprising a key, wherein the staple cartridge is removably positionable in the end effector, and wherein the key is configured to engage the first lockout arm and the second lockout arm to transition the firing lockout to the unlocked state.

Example 35—The surgical stapler of Example 34, wherein the staple cartridge further comprises a sled, and wherein the sled comprises the key.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Pat. No. D974,560, entitled STAPLE CARTRIDGE, which issued on Jan. 3, 2023, U.S. Design Pat. No. D967,421, entitled STAPLE CARTRIDGE, which issued on Oct. 18, 2022, U.S. Design Pat. No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

While several configurations have been described, additional modifications are within the scope of the present disclosure, which is intended to cover any variations, uses, or adaptations of the disclosed configurations using its general principles.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

It is worthy to note that any reference to “one aspect,” “an aspect,” “an exemplification,” “one exemplification,” and the like means that a particular feature, structure, or characteristic described in connection with the aspect is included in one aspect. Thus, appearances of the phrases “in one aspect,” “in an aspect,” “in an exemplification,” and “in one exemplification” in various places throughout the specification are not necessarily all referring to the same aspect. The particular features, structures or characteristics may be combined in any suitable manner in various aspects.

It is worthy to note that any reference numbers included in the appended claims are used to reference exemplary embodiments/elements described in the present disclosure. Accordingly, any such reference numbers are not meant to limit the scope of the subject matter recited in the appended claims.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. A surgical stapler, comprising:
an end effector comprising an elongate channel;
a firing beam;
a firing bar movable to transition the firing beam from a proximal position toward a distal position during a firing stroke, wherein the firing bar comprises a plurality of laminated strips, and wherein the plurality of laminated strips comprises:
a central laminated strip; and
a first subset of laminated strips extending on a first lateral side of the central laminated strip, wherein a first notch is defined in the first subset of laminated strips; and
a firing lockout comprising a first lockout arm extending on a first lateral side of the firing bar, wherein the first lockout arm comprises a first projection; and
wherein the firing lockout is configurable between:
a locked state in which the first projection is received in the first notch to prevent the firing beam from moving toward the distal position; and
an unlocked state in which the first projection is displaced from the first notch to permit the firing beam to move toward the distal position.

17. The surgical stapler of claim 16, wherein the plurality of laminated strips further comprises a second subset of laminated strips extending on a second lateral side of the central laminated strip, and wherein a second notch is defined in the second subset of laminated strips.

18. The surgical stapler of claim 17, wherein the firing lockout further comprises a second lockout arm extending on a second lateral side of the firing bar, and wherein the second lockout arm comprises a second projection.

19. The surgical stapler of claim 18, wherein:
in the locked state of the firing lockout, the second projection is received in the second notch to prevent the firing beam from moving toward the distal position; and
in the unlocked state of the firing lockout, the second projection is displaced from the second notch to permit the firing beam to move toward the distal position.

20. The surgical stapler of claim 18, wherein the firing lockout further comprises a central body portion, and wherein the first lockout arm and the second lockout arm extend from the central body portion.

21. The surgical stapler of claim 20, wherein the firing bar extends above the central body portion.

22. The surgical stapler of claim 16, further comprising a staple cartridge comprising a key, wherein the staple cartridge is removably positionable in the elongate channel, and wherein the key is configured to engage the first lockout arm to transition the firing lockout to the unlocked state.

23. The surgical stapler of claim 22, wherein the first lockout arm comprises a cam surface, and wherein the key is configured to engage the cam surface to transition the firing lockout to the unlocked state.

24. The surgical stapler of claim 22, wherein the staple cartridge comprises a cartridge body, and wherein the cartridge body comprises the key.

25. The surgical stapler of claim 22, wherein the staple cartridge comprises a sled, and wherein the sled comprises the key.

26. The surgical stapler of claim 22, wherein the staple cartridge comprises a retainer, and wherein the retainer comprises the key.

27. The surgical stapler of claim 22, wherein the end effector further comprises an anvil movable relative to the elongate channel between an open position and a closed position, and wherein the firing stroke comprises:
a first stroke portion in which the firing beam moves the anvil toward the closed position; and
a second stroke portion in which the firing beam deploys staples from the staple cartridge.

28. The surgical stapler of claim 16, wherein the firing lockout comprises a lockout spring, and wherein the lockout spring is naturally biased toward the locked state.

29. The surgical stapler of claim 16, wherein the end effector further comprises an anvil, and wherein the firing beam comprises:
a first cam configured to engage the elongate channel during the firing stroke; and
a second cam configured to engage the anvil during the firing stroke.

30. The surgical stapler of claim 16, wherein the firing beam comprises a knife.

31. A surgical stapler, comprising:
an end effector comprising a staple cartridge comprising a plurality of staples removably stored therein;
a firing beam;
a firing bar movable to transition the firing beam from a proximal position toward a distal position during a firing stroke, wherein the firing bar comprises a plurality of laminated strips, and wherein the plurality of laminated strips comprises:
a central laminated strip;
a first subset of laminated strips extending on a first lateral side of the central laminated strip, wherein a first notch is defined in the first subset of laminated strips;
a second subset of laminated strips extending on a second lateral side of the central laminated strip, wherein a second notch is defined in the second subset of laminated strips; and
a firing lockout, comprising:
a body;
a first lockout arm extending from the body on a first lateral side of the firing bar, wherein the first lockout arm comprises a first projection; and
a second lockout arm extending from the body on a second lateral side of the firing bar, wherein the second lockout arm comprises a second projection;
wherein the firing lockout is configurable between:
a locked state in which the first projection is received in the first notch and the second projection is received in the second notch to prevent the firing beam from moving toward the distal position; and
an unlocked state in which the first projection is displaced from the first notch and the second projection is displaced from the second notch to permit the firing beam to move toward the distal position;
wherein the staple cartridge further comprises:
a first key configured to engage the first lockout arm to displace the first projection from the first notch; and
a second key configured to engage the second lockout arm to displace the second projection from the second notch, wherein the first key and the second key transition the firing lockout to the unlocked state.

32. The surgical stapler of claim 31, wherein the staple cartridge further comprises a sled, and wherein the sled comprises the first key and the second key.

33. A surgical stapler, comprising:
an end effector comprising:
a first jaw; and
a second jaw movable relative to the first jaw between an open position and a closed position;
a firing beam;
a firing bar movable to transition the firing beam from a proximal position toward a distal position during a firing stroke, wherein the firing bar comprises a plurality of laminated strips, and wherein an aperture is defined through the plurality of laminated strips; and
a firing lockout, comprising:
a first lockout arm extending on a first lateral side of the firing bar, wherein the first lockout arm comprises a first projection; and
a second lockout arm extending on a second lateral side of the firing bar, wherein the second lockout arm comprises a second projection;
wherein the firing lockout is configurable between:
a locked state in which the first projection and the second projection extend within the aperture to prevent the firing beam from moving toward the distal position; and
an unlocked state in which the first projection and are second projection are displaced from the aperture to prevent the firing beam from moving toward the distal position.

34. The surgical stapler of claim 33, further comprising a staple cartridge comprising a key, wherein the staple cartridge is removably positionable in the end effector, and wherein the key is configured to engage the first lockout arm and the second lockout arm to transition the firing lockout to the unlocked state.

35. The surgical stapler of claim 34, wherein the staple cartridge further comprises a sled, and wherein the sled comprises the key.
